SEQUENCE LISTING (4 sequences)

SEQ ID NO: 1 - protein, 647 aa, Artificial Sequence
  E. coli BL21(DE3)pLysS
MLLINFFIAVLGVISLSPIVVARYILRRDCTTVTVLSSPESVTSSNHVELASHEMCDSTLSASLYIYNDDYDKIVTLYYLTSSGTTGSVTASYSSSLSNNWELWSLSAPAADAVEITGASYVDSDASATYATSFDIPLTTTTTSSSSASATSTSSLTTTSSVSISVSVPTGTAANWRGRAIYEIVTDRFARTDGSTTYLCDVTDRVYCGGSYEGIINMLDYIEGMGFTAIWISPIVENIPDDTGYGYAYHGYWMKDIFALNTNFGTADDLIALATELHNRGMYLMVDIVVNHFAFSGSHADVDYSEYFPYSSEDYFHSFCWITDYSNETNVEQCWLGDDTVPLVDVNTELDTVKSEYQSWVEELIANYSIDGLRIDTVKHVEMDFWAPFEEAAGIYAVGEVFDGDPSYTCPYEENLDGVLNYPVYYPVVSAFESVSGSVSSLVDMIDTLKSECTDTTLLGSFLENEDNPRFPSYTSDESLIKNAIAFTMLSDGIPIIYYGEEQGLNGGNDPYNREALWLTGYSTTSTFYKYIASLNEIRNEAIYKDDTYLTYENWVIYSDSTTIAMRKGFTGNEIITVLSNLGTSGSSYTLTLSNTGYTASSVVYEILTCTAVTVDSSGNLAVPMSSGLPKVFYEESQLVGSGICSM

SEQ ID NO: 2 - DNA, 1946 bp, Artificial Sequence
  E. coli BL21(DE3)pLysS
atgttgctgatcaactttttcatcgctgttctgggagtgatatcactgtctcctattgtggttgctcgttatattcttcgacgagattgcactacagttacggtcttgtcctcccctgagtctgtgacgagttcgaaccatgttcagctagccagtcatgagatgtgcgacagtaccttgtcagcgtccctttatatctacaatgatgattatgataagattgtgacactttattatcttacatcgtcgggcacaactgggtccgtaacagcgtcttattcttctagtttgagtaacaactgggaattgtggtctctctcggctccggctgcagatgctgtcgagatcactggagctagttatgtagacagcgatgcatctgcgacatacgccacgtcttttgatatacctcttactaccacgacaacgtcgtcgtcttctgctagtgcgacttcaacatctagtctaaccacaacatctagtgtttccatttcggtgtccgtccctacaggaacagctgcaaattggcgaggtagggctatctatcagatcgtgactgatagatttgcacgcactgacggctccaccacatatttatgcgatgttaccgatagggtctattgcggagggtcttatcaggggattatcaatatgctggattacatccaaggcatgggctttactgctatttggatttctcctatagtggaaaatattcccgatgacaccggatacggttacgcatatcatggttattggatgaaagatatcttcgccctgaatacaaattttggtactgcagacgatttgatagcgttggctacggaattgcataatcgcggcatgtacttgatggttgatattgttgtcaatcactttgctttctcaggaagtcatgccgacgtggactactctgaatatttcccgtattcgtcccaggattattttcattcattttgctggattacagattactcgaatcagacaaacgttgagcagtgctggcttggcgacgatactgttcctctcgtggacgtcaatacccaacttgacaccgtgaaaagtgaatatcaatcctgggttcaagaacttatagctaattactctattgacggcctaagaattgacaccgtcaagcacgtgcagatggatttttgggcaccatttcaagaggctgcagggatttacgccgttggtgaagtattcgacggtgatccatcctacacatgtccatatcaggaaaatcttgacggtgtcttgaattatcctgtttattatcctgtcgtctctgcgtttgagagtgttagtgggtcggtctcctcgttagtcgatatgattgatacgctcaagtctgaatgcaccgacactactctcctaggctcctttctagagaatcaagataatccgcgattccctagctacacttctgatgagtctttaattaaaaatgcgatcgctttcactatgctctcagacggcattcccataatttattacggtcaggagcaaggcctcaatggtggaaacgatccctataatcgagaggcgctttggcttacgggctactccacaacgtcgacgttctacaaatacattgcgtcgttgaatcagattagaaatcaggctatatacaaagatgatacttatctcacatatcagaactgggttatttattcggattccacgacaatagcaatgcggaaaggttttacagggaaccaaataattacggttctgtcaaatcttgggaccagtggcagttcgtacactttgacgctttcgaatacgggatataccgcatctagcgttgtatatgagatcttgacatgcacagctgtgactgtggattcgtctgggaatttggcagtgccgatgtccagtggcctaccaaaagtcttttatcaggaatcgcaactggttggctctggaatctgctccatgtagag

SEQ ID NO: 3 - DNA, 27 bp, Artificial Sequence
  L. starkeyi primer 1(sense)
tacagttacggtcttgtcctcccctga

SEQ ID NO: 4 - DNA, 21 bp, Artificial Sequence
  L. starkeyi primer 2(antisense)
ctctacatggagcagattcca

(19) United States
(12) Patent Application Publication
(10) Pub. No.: US 2007/0077212 A1
(43) Pub. Date: Apr. 5, 2007

(54) Protein with activity of hydrolyzing amylopectin, starch, glycogen and amylose, gene encoding the same, cell expressing the same, and production method thereof

(75) Inventors: Do-Man Kim, Gwangju (KR); Hee-Kyoung Kang, Gwangju (KR); Jin-Ha Lee, Gwangju (KR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: LIFENZA CO., LTD., Anyang, Kyungki-do (KR)

(21) Appl. No.: 10/588,052

(22) PCT Filed: Jan. 27, 2005
(86) PCT No.: PCT/KR05/00235

§ 371 Date: Jul. 31, 2006

(30) Foreign Application Priority Data
Jan. 30, 2004 (KR) 10-2004-0006186

(51) Int. Cl. A61K 8/96; C12N 9/32; C12N 1/21

(52) U.S. Cl. 424/50; 435/204; 435/252.33

(57) ABSTRACT

Disclosed are an enzyme, having the amino acid sequence of SEQ. ID. No. 1 with the activity of hydrolyzing amylopectin, starch, glycogen and amylose, a gene encoding the enzyme, and a transformed cell expressing the gene. Also disclosed is a method of producing an enzyme capable of degrading amylopectin, starch, glycogen and amylose, which comprises culturing the cell, expressing the enzyme in the cell and purifying the enzyme. A composition comprising the enzyme is provided for removing dextran or polysaccharide contaminants during sugar production.

### BACKGROUND OF THE INVENTION

1. Field of the Invention

The present invention relates to a protein that degrades amylopectin, starch, glycogen and amylose, a gene thereof, an expression cell thereof, and a production method thereof. More particularly, the present invention relates to an enzyme useful not only in anti-plaque compositions or mouthwashes due to its ability to inhibit the formation of dental plaque and degrade previously formed plaque, but also in dextran removal during sugar production due to its excellent ability to hydrolyze dextran, a gene coding for the enzyme, a cell expressing the enzyme, and a method of producing the enzyme.

2. Description of the Related Art

Plaque is a biofilm built up on the teeth, resulting from microbial colonization of the tooth surface. The bulk of dental plaque is composed of bacteria-derived extracellular polysaccharide known as glucan (insoluble glucan), also called mutan, which enhances the colonization. Amounting to about 20% of the dried weight of plaque, this polysaccharide acts as an important factor to cause dental caries. Structural studies of glucans produced by Streptococcus mutans revealed that glucose moieties of the insoluble glucans are linked to each other mainly via α-1,3-, α-1,4-, and α-1,6-D-glucosidic bonds. Effective elimination of plaque, therefore, demands mutanolytic, amylolytic and dextranolytic activities.

Conventionally, the prevention of the formation of plaque and dental caries has mainly depended on the reduction of the growth of Streptococcus mutans (S. mutans) in the mouth. In this regard, compounds with activity against S. mutans growth, such as antiseptics or fluorine, are included in oral products such as toothpastes or mouthwash. Inhibitory as it is of the growth of S. mutans, fluorine, which is a popular anti-tooth cavity compound, gives rise to dental fluorosis (formation of mottles in the dental enamel) as well as causing side effects such as strong toxicity and air pollution. Another attempt has been made to prevent dental caries with enzymes such as dextranase; however, its effect has yet to be proven.

U.S. Pat. No. 5,741,773 provides a dentifrice composition containing glycomacropeptide having antiplaque and anticaries activity. The conventional technique is directed to inhibiting the growth of the bacteria that cause dental caries. However, nowhere are suggested the prevention of plaque formation or the hydrolysis of previously formed plaque.

U.S. Pat. No. 6,485,953 (corresponding to Korean Pat. No. 10-0358376), issued to the present inventors, suggests the use of DXAMase capable of hydrolyzing polysaccharides of various structures in inhibiting the formation of dental plaque and degrading previously formed dental plaque. In addition to an enzyme capable of degrading various polysaccharides, a microorganism (Lipomyces starkeyi KFCC-11077) producing the enzyme and a composition containing the enzyme are also disclosed.

However, there is still a need for an enzyme that has better activity in inhibiting plaque formation as well as hydrolyzing previously formed plaque.

In Korean Pat. Appl'n No. 10-2001-48442, the present inventors also suggested that the enzyme DXAMase produced by the microorganism (Lipomyces starkeyi KFCC-11077) of Korean Pat. No. 10-0358376 can be useful in removing dextran due to its high dextran-degrading activity.

There is therefore a clear need in the art to develop a new enzyme having dextran degradation activity sufficient for dextran removal.

### SUMMARY OF THE INVENTION

Accordingly, the present invention has been made keeping in mind the above problems occurring in the prior art, and an object of the present invention is to provide a novel enzyme capable of hydrolyzing various polysaccharides including amylopectin, starch, glycogen and amylose, and a gene encoding the enzyme.

It is another object of the present invention to provide a strain which carries the gene.

It is a further object of the present invention to provide a method of producing the enzyme and the gene.

It is still a further object of the present invention to provide an industrially useful composition comprising the enzyme.

In accordance with an aspect of the present invention, there are provided a protein, comprising an amino acid sequence of SEQ. ID. No. 1, which hydrolyzes amylopectin, starch, glycogen and amylose, a derivative thereof, or an enzymatic fragment thereof having the activity, and a gene coding for the protein.

In accordance with another aspect of the present invention, there is provided a transformed cell, expressing the gene.

In accordance with a further aspect of the present invention, there is provided a method of producing an enzyme hydrolyzing amylopectin, starch, glycogen and amylose, comprising: culturing the cell; expressing the enzyme in the cultured cell; and purifying the expressed enzyme.

### BRIEF DESCRIPTION OF THE DRAWINGS

The above and other objects, features and advantages of the present invention will be more clearly understood from the following detailed description taken in conjunction with the accompanying drawings, in which:

FIG. 1 shows an amino acid sequence of the carbohydrolase derived from Lipomyces starkeyi (LSA) according to the present invention and a 1946 bp nucleotide sequence encoding the amino acid sequence, wherein a PCR primer, analyzed through the N-terminal amino acid sequencing of a mature protein, for cloning the mature protein in a vector corresponds to underlined normal characters, a splicing site for a signal peptide is indicated by the arrow, and conserved regions of α-amylase are expressed as underlined bold characters;

FIG. 2 is a photograph showing an SDS-PAGE result in which a boiled enzyme (lane 1) and an unboiled enzyme are run on a gel, and a Western blotting result in which an anti-carbohydrolase antibody is conjugated with a boiled enzyme (lane 3);

FIG. 3 is a photograph showing SDS-PAGE and Western blotting results in which the LSA of the present invention indicated by the arrow is electrophoresed along with a molecular weight marker (M) on gels, with visualization performed by coomassie blue staining (lane 1) and by activity staining (lane 2), and is allowed to react with an anti-LSA antibody of the mother cell (lane 3);

FIG. 4 is a graph in which the activity and stability of the LSA of the present invention are plotted versus temperature;

FIG. 5 is a graph in which the activity and stability of the LSA of the present invention are plotted versus pH value;

FIG. 6 is a graph showing the effect of acetone on the activity of the LSA of the present invention;

FIG. 7 is a graph showing the effect of ethanol on the activity of the LSA of the present invention; and

FIG. 8 is a photograph of a TLC result showing the enzymatic activity of the LSA of the present invention in which starch samples (1% w/v) are analyzed, along with maltodextrin (Mn), before and after being hydrolyzed by the enzyme (lanes 1 and 2 in panel A, respectively) and maltooligosaccharide samples (1% w/v) are analyzed after purified LSA is allowed to react with a series of maltooligosaccharides including G1 (glucose) to G7 (maltoheptaose) (lanes 1 to 7 in panel B, respectively).

### DESCRIPTION OF THE PREFERRED EMBODIMENTS

The acquisition of a gene coding for the carbohydrolase (LSA) of the present invention starts by culturing Lipomyces starkeyi in a medium containing starch. Next, on the basis of N-terminal amino acid sequences of carbohydrate hydrolyzing enzymes purified from L. starkeyi, primers comprising expected conserved regions are constructed, followed by PCR with the primers. The PCR product,. approximately 2 kb long, is used for 5′ RACE and 3′ RACE to allow for a complete carbohydrolase gene (LSA). After being amplified by PCR, the gene is cloned in the vector pRSETB (Invitrogen, U.S.A.) with which E. coli BL21(DE)pLysS is then transformed.

L. starkeyi is known to produce endo-dextranase (EC 3.2.1.11) which degrades dextran and x-amylase which degrades starch. This microorganism has been applied to foods and not yet reported to produce antibiotics or other toxic metabolites.

Most of the dextranases produced by microorganisms, except for a few derived from bacteria, are known as inducible enzymes. L. starkeyi ATCC74054, reported first in U.S. Pat. No. 5,229,277, produces both dextranase and amylase whose characteristics are also disclosed. It is also reported that the strain produces low molecular weight dextrans from sucrose and starch. On the basis of the findings, the present inventors have acquired Korean Pat. No. 10-0358376 on Oct. 11, 2002 (corresponding to U.S. Pat. No. 6,485,953 dated Nov. 26, 2002) which relates to a DXAMase enzyme capable of hydrolyzing both dextran and starch, a microorganism producing the enzyme (identified as Lipomyces starkeyi KFCC-11077), and a composition comprising the enzyme.

The enzyme expressed from the gene (lsa) of the present invention is a carbohydrolase capable of hydrolyzing amylopectin, starch, glycogen and amylose. Also, the enzyme according to the present invention is found to degrade dextran, alpha-cyclodextrin and pullulan. The enzyme is highly stable. Not only is its activity 90% of its maximum over a relatively broad pH range (pH 5-8), but also it is not inhibited even by a denaturation solution such as an EGTA-containing solution. Ca²⁺or Mg²⁺serves as a cofactor for the enzyme.

Also, the present invention is directed to a novel microorganism carrying the gene coding for the carbohydrolase. The strain E. coli BL21(DE3)pLysS according to the present invention was deposited in the Korean Collection for Type Cultures (KCTC) located in Yusung Gu, Daejeon City, South Korea, with the accession number of KCTC10573BP, on Dec. 24, 2003.

Also, the present invention is directed to a method of producing the carbohydrolase. First, the strain E. coli BL21(DE3)pLysS is cultured. After being harvested from the culture, the cells are disrupted using glass beads to isolate the carbohydrolase therefrom.

A composition comprising the enzyme of the present invention may be used in a variety of oral care applications. By virtue of its ability to degrade polysaccharides such as dextran and amylose, the enzyme of the present invention is also effectively used to remove dextran during sugar production. Additionally, compositions comprising the enzyme according to the present invention can be applied to foods such as gum, drinks, milks, etc. and their constituents may be readily determined by those who are skilled in the art.

A better understanding of the present invention may be obtained through the following examples which are set forth to illustrate, but are not to be construed as the limit of the present invention.

### EXAMPLE 1

### lsa gene cloning in Lipomyces starkeyi

1) Strain and plasmid

Lipomyces starkeyi KFCC 11077, which produces DXAMase having dextranase and amylase activity, was used as a DNA donor for cDNA isolation and amylase gene selection. General DNA manipulation and DNA sequencing were carried out with Escherichia coli DH5α and pGEM-T easy (Promega, USA). For the construction of a cDNA library, E. coli XL1-Blue and SOLR (Stratagene, USA) were used as host cells with lambda phase Uni-ZAP XR (Stratagene, USA) as a vector.

2) Culture condition

L. starkeyi was cultured in an LW medium supplemented with 1% (w/v) starch. The LW medium, containing 0.3% (w/v) yeast extract and 0.3 (w/v) KH₂PO₄, was adjusted to pH 4.5 with HC1. For bacterial culture, LB (1% trypton, 0.5% yeast extract, 1% NaCl, pH 7.3) and LBA (LB containing 50 g ampicillin/ml) were used.

3) Purification of carbohydrolase

To obtain a preculture, L. starkeyi was grown in an LW medium supplemented with 1% (w/v) starch, with shaking. Afterwards, the preculture was cultured in a 10 L fermentor (Hanil R&D, Korea) containing 8.3 liters of an LW medium supplemented with 1% (w/g) starch as a carbon source to produce the carbohydrolase of interest. The culture supernatant was filtered through a 100K cut-off hollow fiber (Saehan, Korea) and then concentrated to 830 ml through a 30K cut-off hollow fiber (Millipore, USA). By the addition of ammonium sulfate (Sigma Chemical Co., USA) in an amount of up to 70% of the amount of the concentrate, proteins were precipitated. After being -centrifuged, the precipitates were suspended in 60 ml of a 20 mM potassium phosphate buffered solution (pH 6.4). The concentrations and titers of the protein were measured at every purification stage. The protein concentrate (30 mg/1.5 ml) was loaded onto a DEAE-Sepharose column equilibrated with a 20 mM potassium phosphate buffered solution, followed by elution with a concentration gradient from 0 to 1.0 M of NaCl. Active elute fractions were pooled, concentrated, and loaded onto a GPC column (Bio-Rad Co., A-0.5 m, 70 cm×2.6 cm) to isolate the protein of interest. The column was equilibrated with 50 mM citrate phosphate buffered solution (pH 5.5) and the concentrate contained proteins in an amount of 4 mg/ml.

4) Isolation of poly A+RNA

L. starkeyi was inoculated into an LW medium supplemented with 1% (w/v) starch. After culturing at 28° C. for 36 hours (to the mid-exponential growth phase), the culture was centrifuged at 6,500×g to harvest a cell pellet. Total RNA was isolated using :glass beads and hot acid phenol. Cells were mixed with a solution containing guanidine thiocyanate, 0.5% sodium lauryl sarcosinate, 0.1M β-mercaptoethanol, and 25 mM sodium citrate (pH 7.0), and then combined with equal volumes of acid-washed glass beads and a mixture of phenol/chloroform/isoamylalcohol (25/24/1, v/v/v), followed by being vortexed for 5 min at the highest speed. After centrifugation, the mixed solution was mixed with three volumes of isopropanol and 0.3 volumes of 3M sodium acetate to produce an RNA pellet which was then dissolved in Rnase-free distilled water for storage until next use.

5) NH₂-terminal amino acid sequencing and oligonucleotide synthesis

The NH₂-terminal amino acid sequence of purified amylase was analyzed using an automated protein sequencer (Model 471A, Applied Biosystems, USA) based on the Edman degradation method.

After being purified, the carbohydrolase (LSA, having dextranase and amylase activity) obtained from L. starkeyi was analyzed to the N-terminal amino acid sequence DXSTVTVLSSPETVT (wherein X remained unrevealed). On the basis of the amino acid sequence TVTVLSSPE, an oligonucleotide, that is, a sense primer 1 (5′-TACAGTTACGGTCTTGTCCTCCCCTGA-3′) (SEQ. ID. NO. 3) was designed. An antisense primer 2 (5′-CTCTACATGGAGCAGATTCCA-3′) (SEQ. ID. NO. 4) was constructed. The PCT product obtained with the sense and antisense primers was found to have a size of about 2 kb as measured by electrophoresis.

6) Construction of L. starkeyi cDNA library

From 5 g of the poly (A)+RNA obtained by culturing for 36 hours in a starch-added medium, cDNAs were prepared using ZAP-cDNA synthesis kit. 500 kb or longer sizes of the prepared cDNAs were separated by a spin column fraction method and ligated with a Uni-ZAP XR vector which had been digested with EcoRI-XhoI. The in vitro packing of ligated phage cDNAs was performed with a Gigapack Gold kit (Stratagene, USA).

7) lsa cloning

A DNA fragments having the open leading frame of the gene lsa was obtained by PCR with a pair of primers: a sense primer 5′-TACAGTTACGGTCTTGTCCTCCCCTGA-3′ and an antisense primer 5′-CTCTACATGGAGCAGATTCCA-3′ which respectively correspond to N-terminal and C-terminal amino acid sequences of the protein showing dextranase and amylase characteristics. After being separated on agarose gel, the PCR product was purified with an AccPrep™ gel extraction kit (Bioneer, Korea) and ligated with pGEM-T easy vector (Promega, USA). Base sequencing was performed using ABI PRISM Cycle Sequencing Kit (Perkin Elmer Corp. USA) in a GeneAmP 9600 thermal cycler DNA sequencing system (Model 373-18, Applied Biosystems, USA).

8) Heterologous expression and purification of LSA protein in E. coli

The gene lsa was inserted into the SacI-EcoRI site of pRSETB vector (Invitrogen USA) to prepare a recombinant vector pRSET-LSA. E. coli BL21(DE3)pLysS transformed with pRSET-LSA was cultured at 37° C. to a midstationary phase in an LB medium containing 50 mg/l ampicillin. After the addition of IPTG to the culture to a final concentration of 1 mM, incubation was carried out at 28° C. for 6 hours. Cells were harvested by centrifugation (5000 g×10 min), washed with 0.1 M potassium phosphate (pH 7.4 and lyzed by sonication. Purification of the expressed protein was performed with Ni²⁺-nitrilotriacetic acid-agarose (NTA) (Quiagene, Germany). The cell lysate was combined with Ni²⁺-NTA and allowed to stand for 1 hour at 4° C., and the mixture was loaded onto a column which was then washed four times with a washing buffer. Each 0.5 ml of the protein fraction was emulsified with a buffer.

9) Electrophoresis and activity staining

For sodium dodecyl sulfate-polyacrylamide gel electrophoresis (SDS-PAGE), protein samples were loaded onto 10% polyacrylamide gel in Tris-glycine buffer (pH 8.8). The polyacrylamide gel contained 1% starch in order to detect whether the protein samples could degrade starch polysaccharide. After completion of the electrophoresis, SDS was removed by washing the gel with 50 mM Tris-HCl buffer (pH 8.0) and a 20% 2-propanol solution for 1 hour. The gel was immersed in a reaction buffer (50 mM sodium acetate, 5 mM CaCl₂, pH 5) at 37° C. for 2 days and then in an iodide solution (0.3% iodide, 3% potassium iodide) for 10 min, and washed with distilled water. The starch hydrolyzing activity was identified by the appearance of a clear zone against a brown background.

To determine the molecular weight of the protein of interest, a marker including myosin 200 kDa, β-galactosidase (116 kD), phosphorylase b (97.4 kDa), serum albumin (66.2 kDa), carbonic anhydrase (31 kDa) and aprotinin (6.5 kDa) was also run on the gel.

10) Western blotting

Following the electrophoresis, the protein in the gel was transferred to a PVDF membrane in the presence of an electric field. LSA was detected using rabbit polyclonal antibodies specific for carbohydrolase (having both dextranase and amylase activity). The serum containing anti-carbohydrolase antibodies was diluted in a ratio of 1:200 for use. The membrane treated with the antibodies was washed three times with Tris-buffered saline (TBS) (20 mM Tris-HCl, 137 mM NaCl) containing 0.1% Tween 20(T). Antigen-antibody conjugates were probed with the ECL Western blotting analysis system (Amersham Pharmacia, USA) in combination with a secondary antibody. Peroxidase-conjugated anti-rabbit-IgG (Amersham Pharmacia, USA), serving as the secondary antibody, was diluted in a ratio of 1:1500. Biomax film (Kodak, USA) was used for screen exposure for 1 min.

### EXAMPLE 2

### Assay for Carbohydrolase Activity

The reducing value of the carbohydrolase was determined by a DNS (3,5-dinitrosalicylic acid) method in combination with a copper-bicinchoninate method. That is, 100 μl of copper-bicinchoninate was added to 100 μl of an enzyme solution, and allowed to react at 80C. for 35 min, followed by being cooled for about 15 min. Absorbance was measured at 560 nm.

### EXAMPLE 3

### Assay for Optimal pH and Temperature and Stability of Enzyme

The enzyme LSA was assayed for optimal pH by measuring reaction rates in the range of pH 3-9 at intervals of pH 1.0. For this purpose, 2OmM citrate phosphate buffer (pH 4.0), citrate/phosphate buffer (pH 5-6) and sodium phosphate buffer (pH 7-9) were used. After reaction at 37° C. for 48. hours, the carbohydrolase activity of the enzyme was determined by a DNS method. Also, the pH stability of the enzyme was measured after the enzyme was added to each buffer and allowed to stand for 3 hours at 22° C.

The optimal temperature of the enzyme was determined by measuring the reaction rates of the enzyme which had been allowed to stand for 30 min at various temperatures (20-80° C., 10C. interval). For the determination of temperature stability, the enzyme was measured for residual activity after being allowed to stand for 30 min at various temperatures (20-90° C., 10° C. interval). 1% (w/v) starch was used as a substrate in determining the activity and stability of the enzyme.

### EXAMPLE 4

### Effect of Metal Ions, Chelating Solutions and Denaturizing Solutions on Enzyme Activity

Effects of EDTA, EGTA and metal ions on enzyme activity were measured. EDTA and EGTA were each used at a final concentration of 1 mM. As for metal ions, they included ZnCl₂, CuS0₄, CaCl₂ and MgCl₂ and were used at a final concentration of 5 mM. The enzyme activity was also measured in the presence of dodesyl sulfate (SDS, 0.1%, 0.5%, 1%, 2%), urea (2M), acetone (0-80%) and ethanol (0-70%). For the measurement, the enzyme was allowed to react at 37° C. for 30 min with 2% starch as a substrate.

### RESULTS

Cloning of the gene lsa from Lipomyces starkeyi

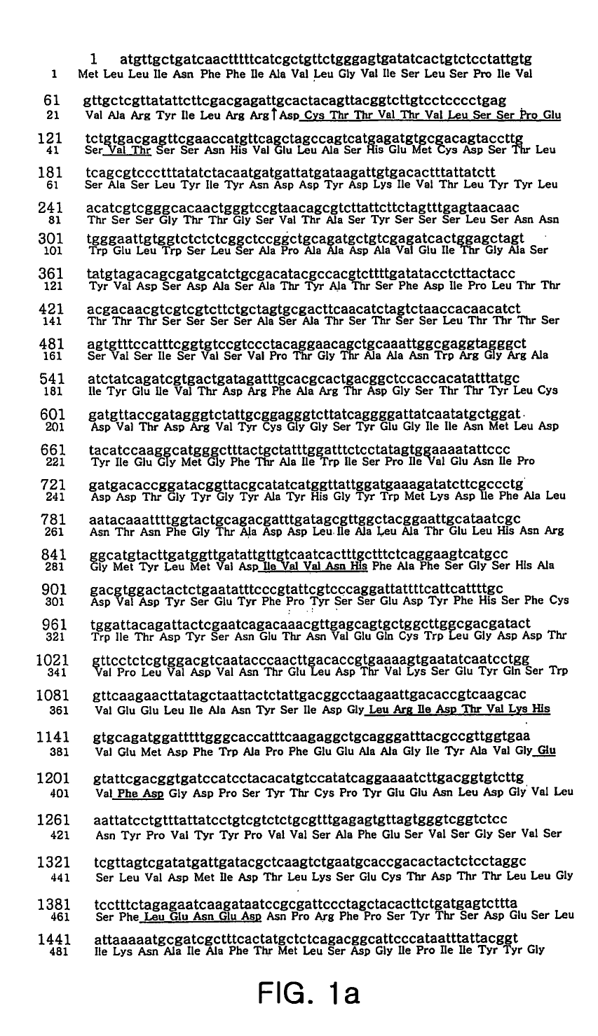

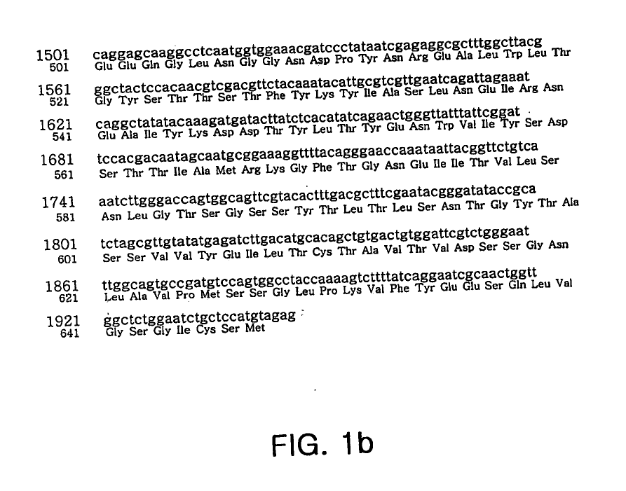

After being purified, the carbohydrolase (LSA) (having both dextranase and amylase activity) derived from L. starkeyi was analyzed to have an N-terminal amino acid sequence of DXSTVTVLSSPETVT (X: an amino acid residue yet not. revealed). On the basis of the amino acid sequence of TVTVLSSPE, a sense primer 1 (5′-TACAGTTACGGTCTTGTCCTCCCCTGA-3′) was designed and synthesized. Separately, an antisense primer 2 (5′-CTCTACATGGAGCAGATTCCA-3′) was constructed. Electrophoresis showed a 2 kb band for the PCR product. Amino acid and base sequencing results are given in FIG. 1 and SEQ. ID. Nos. 1 and 2.

Characterization of lsa gene

From L. starkeyi KFCC 11077 which produces dextranase and amylase, the gene coding for LSA was cloned as a 1946 bp cDNA fragment. In the case of the cDNA fragment, the open reading frame consists of 1944 bp (647 amino acids) with a molecular weight of 71,889 Da, which corresponds to an unmodified LSA precursor. Its mature protein was found to have 619 amino acids (1,857 bp) with a molecular weight of 68,709 Da. It is inferred that the precursor protein is processed at the position between Arg²⁸ and Asp²⁹ so as to make the mature protein (FIG. 1).

The LSA ORF starts with the starting codon ATG at nucleotide position 1 and terminates with the stop codon TAG at nucleotide position 1944. The putative LSA amino acid sequence shares homology with α-amylase derived from various yeasts and plants, cyclodextrin glucanotransferase, pullulanase and α-glycosidase from bacteria, and β-amylase from B. polymyxa. LSA was found to show 52-78% homology with α-amylase of L. kononenkoae, Sw. occidentalis (AMYL) and Sh. fibuligera (ALP1) (Park, J. C., Bai, S., Tai, C. Y. and Chun, S. B. (1992) Nucleotide sequence of the extracellular-amylase gene in the yeast Schwanniomyces occidentailis ATCC 26077. FEMS Microbiol Lett. 93, 17-24; Steyn, A. J. C., Marmur, J. and Pretorius, I. S. (1995) Cloning, sequence analysis and expression in yeasts of a cDNA containing a Lipomyces kononenkoae α-amylase-encoding gene. GENE. 166, 65-7; Ito, T., Yamashita, I. and Fukui, S. (1987) Nucleotide sequence of the α-amylase gene (ALPL) in the yeast Saccharomycopsis fibuligera. FEBS Lett. 219, 339-342). For comparison, four conserved regions among various amylases, including the LSA gene obtained according to the present invention, are given in Table 1, below. Six boxed amino acid residues are identical in the conserved regions.

TABLE 1

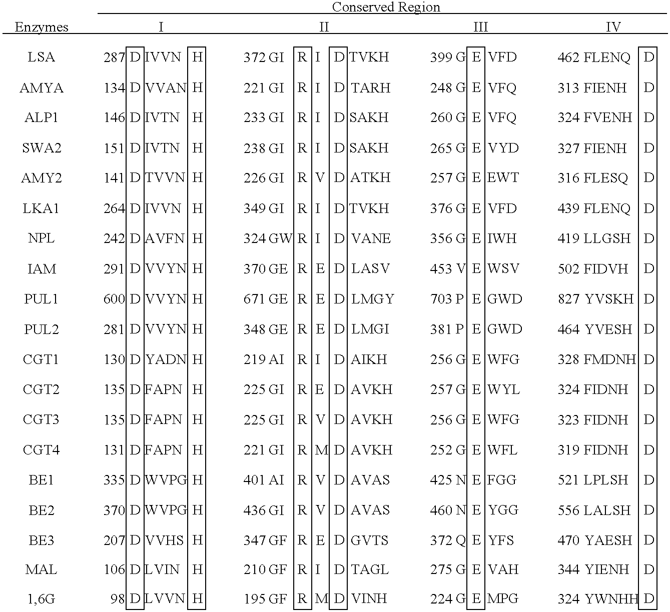

Enzyme Abbr.: LSA, Lipomyces starkeyi α-amylase; AMYA, Aspergillus nidulans α-amylase; ALP1, Saccharomycopsis fibuligera α-amylase; SWA2, Debaromyces occidentails α-amylase; AMY2, Schizosaccahromyces pobme α-amylase; LKA1, L. kononenkoae α-amylase; NPTL, Bacillus stearothermophilus neopulluanase; IAM, Pseudomonas amyloderamosa isoamylase; PUL1, Klebsiella aerogenes pullulanase; PUL2, B. stearothermophilus pullulanase; CGT1, K. pneumoniae cyclodextrin glucanotransferase; CGT2, Paenibacillus macerans cyclodextrin glucanotransferase; CGT3, alkaliphilic Bacillus sp. cyclodextrin glucanotransferase; CGT4, B. stearothermophilus cyclodextrin glucanotransferase; BE1, Escherichia coli branching enzyme; BE2, Synechococcus sp. branching enzyme; BE3, Maize branching enzyme; MAL, Saccharomyces carisbergensis maltase; 1,6G, B. cereus oligo-1,6-glucosidase.

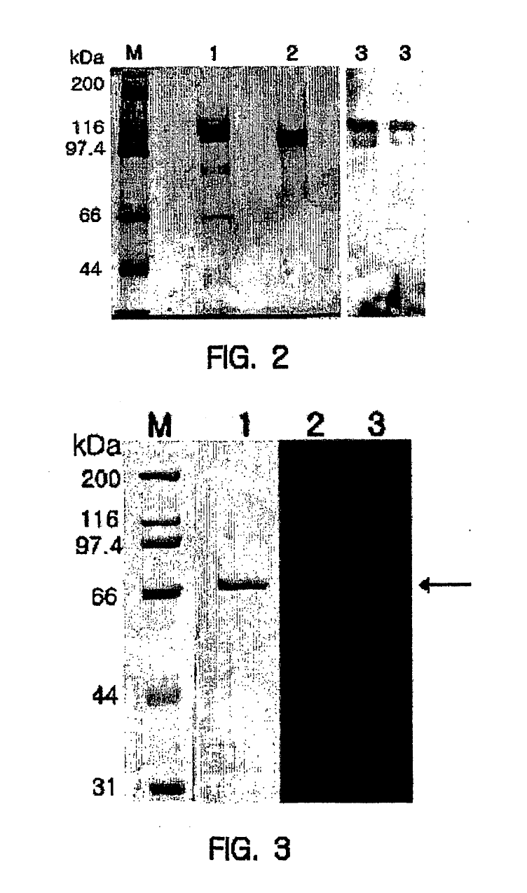

One intron, found between base 966 and 967 in the cDNA, consisting of 60 bases (5′-GTGGTATGTATCTAAGCATATTTG TAGCATTCTATCTTGGAACTGACCGGCCCTCAGTGC-3′) is present in the genomic DNA of LSA. The recombinant LSA prepared according to the present invention was found to differ in molecular weight from the LSA (about 100 kDa) of the mother cell (Lipomyces starkeyi) as measured by SDS-PAGE. This difference is believed to be due to the fact that the enzyme of the mother cell is glycosylated with glycoproteins produced in the yeast. In the case of the carbohydrolase of the mother cell, an anti-carbohydrolase antibody detected approximately 100 kDa (FIG. 2). Because it tends to aggregate with others, an active LSA enzyme, when not boiled, was found to be 200 kDa as measured by gel permeation chromatography.

Expression of lsa gene

Following IPTG induction in E. coli, the cells were harvested, and disrupted by sonication. Proteins were purified using His-tagged affinity column and analyzed by SDS-PAGE (10%) (FIG. 3, lane 1). The band for the mainly expressed protein corresponded to 73 kDa (LSA+His-tag). To examine the ability of the purified protein to degrade polysaccharides such as starch, electrophoresis was carried out using a PAGE gel containing starch. After completion of the electrophoresis, the gel was allowed to stand for 30 min at 37° C. and stained with an iodine solution. A clear zone of LSA activity bands stood out against a brown background (FIG. 3, lane 2). As seen in the Western blotting analysis, the anti-carbohydrolase antibody detected a protein at approximately 73 kDa (LSA+His-tag) (FIG. 3, lane 3).

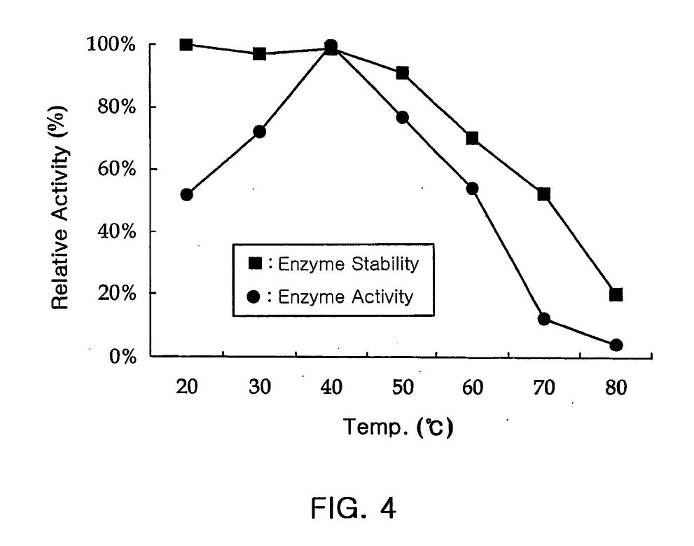

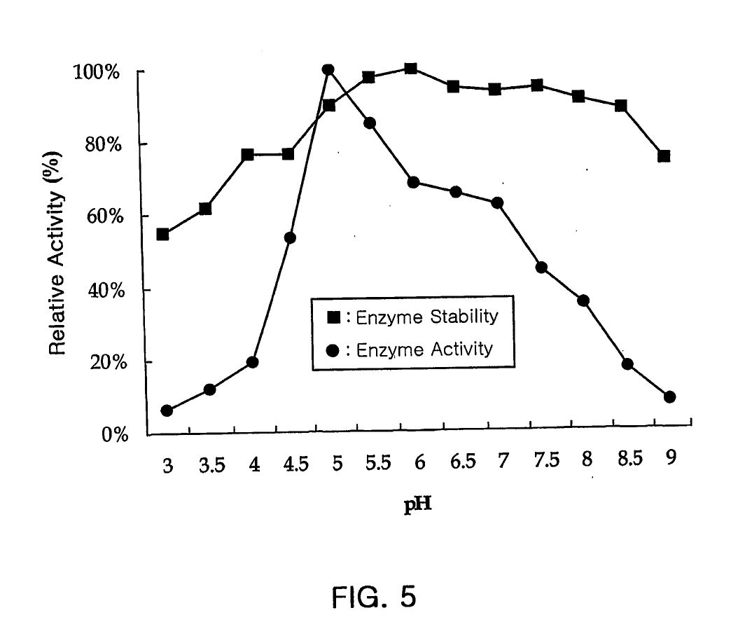

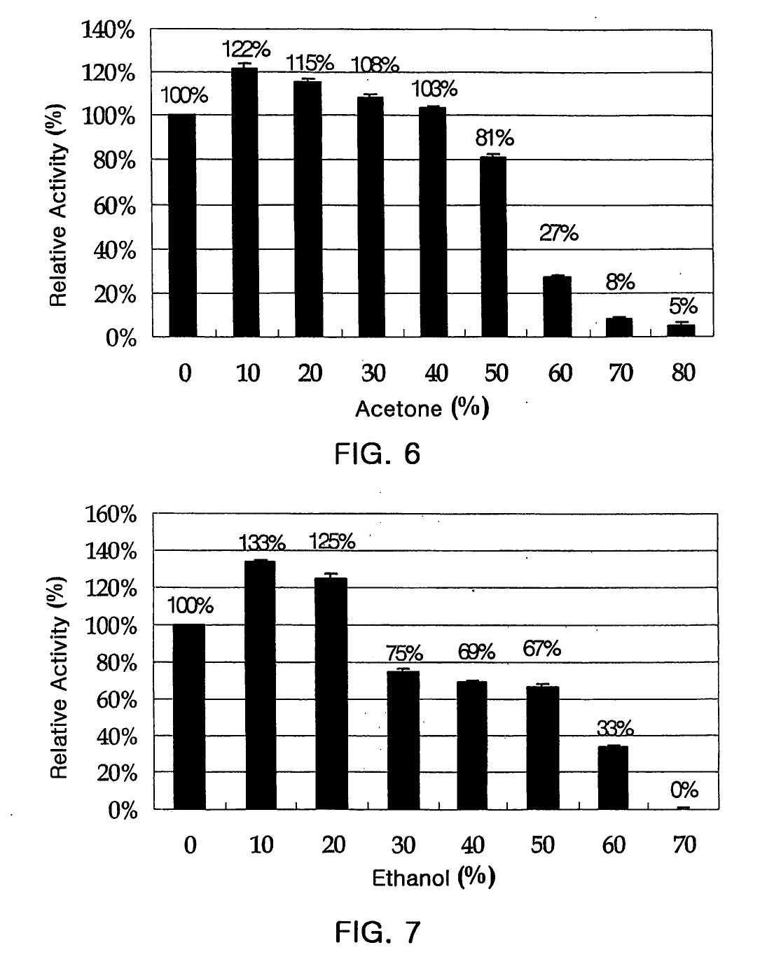

Biochemical characteristics of LSA The LSA enzyme was found to show optimal activity at 40° C., and keep stability in the temperature range of 20-50° C. After incubation at 60° C. for 3 hours, the LSA enzyme was 70% as active as at the stable temperatures (FIG. 4). The amylase activity of the LSA enzyme was kept stably in the pH range of 5-8, with an optimum at pH 6 (FIG. 5).

Whereas it was inhibited by 5 mM Cu²⁺, the starch degradation activity of the enzyme increased by about 315% and 220% in the presence of 5 mM Ca+and 5 mM Mg²⁺, respectively (Table 2). The activity of-the enzyme was inhibited by 1 mM EDTA, but not influenced by 1 mM EGTA. SDS completely inhibited the starch degradation activity of the enzyme, which was increased by urea or acetone. When used in a 10-40% acetone and a 10-20% ethanol solution, the LSA enzyme was increased in activity 1.03-1.22 fold and 1.25-1.33 fold, respectively. In the presence of 60% acetone or ethanol, the LSA enzyme showed lower than 50% of the optimal activity (FIGS. 6 and 7). The high stability of the LSA enzyme of the present invention is quite different from that of starch-hydrolyzing enzymes known thus far.

TABLE 2
Effect of metal ions, chelating agents and denaturants on LSA
enzyme activity

| | | Relative |
|---|---|---|
| Additives | Conc. | Activity (%) |
| None | — | 100 |
| CaCl₂ + EDTA | 5 mM/1 mM | 185 |
| CaCl₂ | 5 mM | 315 |
| CuSO₄ + EDTA | 5 mM/1 mM | 12 |
| CuSO₄ | 5 mM | 20 |
| MgCl₂ + EDTA | 5 mM/1 mM | 129 |
| MgCl₂ | 5 mM | 220 |
| EGTA | 1 mM | 105 |
| EDTA | 1 mM | 46 |
| SDS | 2% | 12 |
| | 1% | 24 |
| | 0.5% | 49 |
| | 0.1% | 66 |
| Urea | 2M | 115 |
| Acetone | 30% | 115 |
| | 20% | 122 |

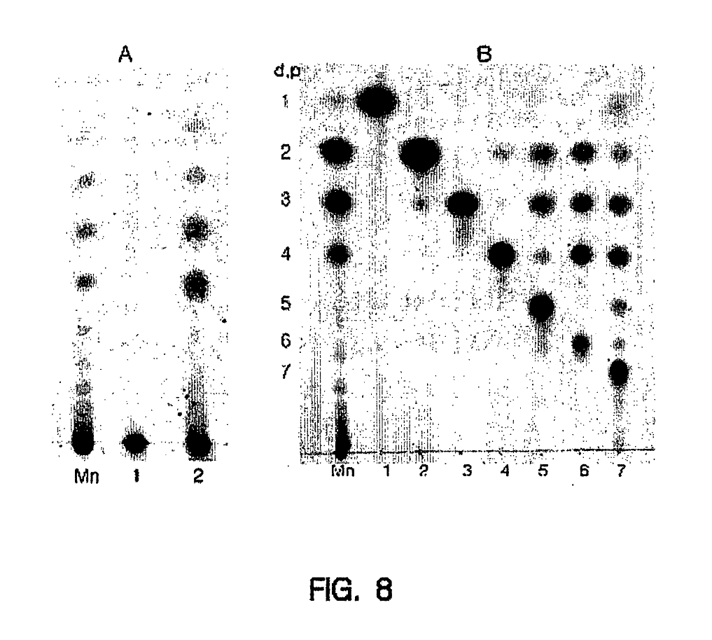

In the early stage of the reaction of LSA with 2% starch, oligosaccharides larger than maltopentaose were produced. Subsequently, the malto oligosaccharides were degraded into maltopentaose and lower oligosaccharides. Finally, maltotriose and maltotetraose were predominant over 10 other oligosaccharides (FIG. 8). When reacted with a mixture of the maltooligosaccharide series (maltose to maltoheptaose), the LSA did not hydrolyze G2 and G3, but degraded G4 into G2, G5 into G2+G3, G6 into G2+G4 or into G3+G3, and G7 into G3+G4 (FIG. 8B). Also, the LSA was found to degrade amylopectin, 15 starch (soluble) and glycogen strongly and amylose, amylodextrin, dextran, a-cyclodextrin and pullulan weakly

TABLE 3
Relative Substrate Specificity of LSA Enzyme

| | Relative |
|---|---|
| Substrate | Activity (%) |
| Starch | 100 |
| Amylopectin | 141 |
| Glycogen | 80 |
| Amylose | 41 |
| Amylodextrin | 17 |
| Dextran | 4 |
| alpha-CD | 4 |
| Pullulan | 3 |

As described above, the enzyme provided by the present invention is able to effectively hydrolyze a variety of polysaccharides, such as amylopectin, starch, glycogen and amylose. With such degradation activity, the enzyme of the present invention not only finds various applications in the dental care industry, including anti-plaque compositions and mouthwashes, but also is useful in removing dextran or polysaccharide contaminants during sugar production.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A protein, comprising an amino acid sequence of SEQ. ID. No. 1, which has the activity of hydrolyzing amylopectin, starch, glycogen and amylose, a derivative thereof, or a fragment thereof.

2. A gene of SEQ. ID. No. 2, encoding the protein, the derivative, or the fragment of claim 1, a derivative thereof, or a fragment thereof.

3. A transformed cell, expressing the gene, the derivative, or the fragment of claim 2.

4. The transformed cell as defined in claim 2, wherein the cell is prokaryotic or eukaryotic.

5. The transformed cell as defined in claim 4, wherein the cell is Escherichia coli DH5@pRLSA deposited with the accession number of KCTC 10573BP.

6. A method of producing an enzyme having activity of hydrolyzing amylopectin, starch, glycogen and amylose, comprising:
culturing the cell of claim 3;
expressing the enzyme in the cultured cell; and
purifying the expressed enzyme.

7. An enzyme, produced by the method of claim 6.

8. A composition, comprising the enzyme of claim 7.

9. The composition as defined in claim 8, wherein the composition is used for dextran removal during sugar production.

10. The composition as defined in claim 8, wherein the composition is used for plaque elimination or as a mouthwash.
